(19) United States
(12) Patent Application Publication
(10) Pub. No.: US 2010/0124577 A1
(43) Pub. Date: May 20, 2010

(54) COMPOSITION FOR THE TREATMENT OF THE HAIR ROOTS

(75) Inventors: Yousef Abdullatif Jamel, Wien (AT); Rafida Mohammed Ahmed Al-Dabooni, Sharjha (AE); Karam Mashaal Hamed al Sari, Sharjha (AE)

Correspondence Address: BUCHANAN, INGERSOLL & ROONEY PC, POST OFFICE BOX 1404, ALEXANDRIA, VA, 22313-1404, US

(73) Assignee: Auron Investments ltd., Hamilton (BM)

(21) Appl. No.: 12/325,867

(22) Filed: Dec. 1, 2008

(30) Foreign Application Priority Data
Nov. 18, 2008 (GB) 0821071.8

(51) Int. Cl. A61K 36/28; A61Q 7/00

(52) U.S. Cl. 424/764

(57) ABSTRACT

The present invention is directed to a composition for the treatment of the hair roots in order
to prevent or at least to reduce the loss of hairs,
to restore the growth of the hairs and
to prevent or at least to reduce the formation of white hairs.

This composition contains an extract of at least one Chamomilla plant and/or of at least one Achillea plant.

### FIELD OF THE INVENTION

The present invention is directed to a composition for the treatment of the hair roots in order
to prevent or at least to reduce the loss of hairs,
to restore the growth of the hairs and
to prevent or at least to reduce the formation of white hairs.

The present invention is also directed to the use of a composition containing an extract of at least one Chamomilla plant and/or of at least one Achillea plant for the preparation of a medicament or of a cosmetic for the treatment of the hair roots in order
to prevent or at least to reduce the loss of hairs,
to restore the growth of the hairs and
to prevent or at least to reduce the formation of white hairs.

Furthermore, the present invention is directed to a method for the treatment of the human or animal body suffering under hair root problems, such as loss of hairs and formation of white hairs.

### BACKGROUND OF THE INVENTION

The therapeutic properties of various plants are known for millennia. Even at the moment when the present application has been filed the nature of the effective components and their properties are in many cases only little understood.

Among the many plants used for therapeutic purposes Chamomilla plants are quite common.

There exists a great variety of Chamomilla plants which belong to the family of the Asteraceae. A part of the Asteraceae family is the genus Anthemis.

Anthemis nobilis L. and Matricaria recutita L. have found wide-spread therapeutic applications. It is often only a specific part of the Chamomilla plants or even an extract thereof which is of interest in therapeutic treatment of a specific disorder and/or of a disease. Such extracts contain the active ingredients of a plant or of a part thereof, often a mixture of the essential oils.

Chamomilla oils can be extracted from the flowers of the Chamomilla plants by steam distillation. Anthemis nobilis L. yields about 1.7 v/m % of essential oil, and Matricaria recutita L. yields about 0.2-0.4 v/m % of essential oil.

The essential oil of Roman chamomile (Matricariae romanae aetheroleum) is produced from Anthemis nobilis L. (Synonym: Chamaemelum nobile (L.) All.). The essential oil of Roman chamomile has a sweet, apple-like fragrance and a very light, clear blue colour. Roman chamomile is cultivated for example in Germany, France, Spain, Italy and Morocco.

The major chemical components of Roman chamomile oil are α-pinene, camphene, β-pinene, sabinene, myrcene, 1,8-cineole, γ-terpinene, caryophyllene, propyl angelate and isobutyl angelate.

The main therapeutic properties of Roman chamomile oil are the following: antiseptic, anti-inflammatory, bactericidal, carminative, sedative, digestive and vulnerary (Hunnius, Pharmazeutisches Wörterbuch, 7. Auflage; 309; Walter de Gruyter; Berlin; New York 1993 and R. Hänsel, O. Sticher, Pharmakognosie-Phytopharmazie, 8. Auflage, 1066-1068; Springer Medizin Verlag Heidelberg 2007).

The essential oil of German chamomile (Matricariae aetheroleum) is produced from Matricaria recutita L. (Synonym: Chamomilla recutica (L.) Rauschert). The essential oil of German chamomile has a sweet, straw-like fragrance and a dark blue colour. German chamomile is cultivated for example in Eastern Europe, in particular Hungary, France and Egypt. The major chemical constituents of German chamomile oil are chamazulene, α-bisabolol, bisabolol oxide A, bisabolol oxide B and bisabolone oxide A.

The main therapeutic properties of German chamomile oil are the following: anti-inflammatory, bactericidal, carminative, digestive, sedative, stomachic and vulnerary (Hunnius, Pharmazeutisches Wörterbuch, 7. Auflage; 309; Walter de Gruyter; Berlin; New York 1993 and R. Hänsel, O. Sticher, Pharmakognosie-Phytopharmazie, 8. Auflage, 1132-1133; Springer Medizin Verlag Heidelberg 2007).

It is an object of the present invention to provide a composition for the treatment of the hair roots in order
to prevent or at least to reduce the loss of hairs,
to restore the growth of the hairs and
to prevent or at least to reduce the formation of white hairs.

It is an object of the present invention to provide a composition comprising the essential oil of Chamomilla, preferably the essential oil of Matricaria recutita L. (Matricariae aetheroleum) and/or of the essential oil of Anthemis nobilis L. (Matricariae romanae aetheroleum) for the treatment of the hair roots in order
to prevent or at least to reduce the loss of hairs,
to restore the growth of the hairs and
to prevent or at least to reduce the formation of white hairs.

It is a further object of the present invention to provide a method for the treatment of the human or animal body suffering under hair root problems, such as loss of hairs and formation of white hairs.

It is also an object of the present invention to provide the use of said composition for the treatment of the human or animal body suffering under hair root problems, such as loss of hairs and formation of white hairs.

These objects are attained with the present invention.

The invention is characterized by the characteristics as defined in the independent claims.

Preferred embodiments are defined in the dependent claims.

### BRIEF DESCRIPTION OF THE INVENTION

It has now surprisingly been found that an extract of at least one Chamomilla plant and/or of at least one Achillea plant, especially an extract comprising the essential oils of the Chamomilla plant and/or of the Achillea plant, obtainable for example by steam distillation, is effective,
to prevent or at least to reduce the loss of hairs,
to restore the growth of the hairs and
to prevent or at least to reduce the formation of white hairs.

This extract exhibits a melanogenesis stimulating activity.

It was found quite surprisingly, that the treatment of patients suffering under hair root problems, such as loss of hairs and formation of white hairs, with a composition according to the present invention resulted in a reduction of the loss of hairs, in a restoration and in an increase of the growth of the hairs in their original colour.

### DETAILED DESCRIPTION OF THE INVENTION

The present invention is based on data obtained using the essential oil of chamomile flower heads, preferably obtainable by steam distillation. Precisely the essential oil is composed of the volatile oil components of the flower heads of Roman chamomile (Anthemis nobilis L.).

Other suitable Chamomilla plants are Anthemis species, such as Anthemis arvensis L., Anthemis cotula L. and Anthemis tinctoria L.

Other suitable plants are Matricaria recutita L., Matricaria discoidea DC, Ormenis multicaulis Br.-BI. & Maire and Eriocephalus punctulatus DC.

The essential oil or oils may be obtained by any suitable procedure known to those of ordinary skill in the art. The essential oil or oils may be obtained for example by using an aqueous or organic extraction medium or by using a water steam distillation.

The essential oil or oils may be separated from other components by means of filtration, chromatography, supercritical fluid extraction etc.

The compositions according to the present invention can be formulated by methods known to those skilled in the art. Pharmaceutically acceptable auxiliary agents can be used.

The compositions according to the present invention are preferably in a form which is suitable for topical application, especially shampoos, hair tonics, conditioners, pomades, lotions, sprays, liniments, creams, ointments, jellies, solutions, suspensions or patches.

The compositions according to the present invention may also be in a form which is suitable for oral application, especially in the form of a capsule.

The exact dosage and frequency of administration depend on several factors. For example it was found that the topical application of a liniment, containing 25% of the essential oil of Anthemis nobilis L., once a day on the scalp of a patient resulted in a reduction of the loss of hairs, in a restoration and in an increase of the growth of the hairs in their original colour.

In the following part are described possible embodiments of the present invention.

The following examples illustrate the present invention.

### EXAMPLES

### Example 1

### Liniment with a Composition of 1 Essential Oil

437.50 ml Olivae oleum raffinatum (PhEur) and 312.50 ml Amygdalae oleum raffinatum (PhEur) were mixed together. 250.00 ml Matricariae romanae aetheroleum (CAS No.: 8015-92-7) were subsequently added under gentle stirring to the mixture of Olivae oleum raffinatum (PhEur) and Amygdalae oleum raffinatum (PhEur) until a clear homogeneous liniment of 1000 ml was obtained.

### Example 2

### Therapeutic Example Using the Liniment from Example 1

The patient was male, 56 years old and had completely white head hairs.

His head hairs began to become gradually white when he was 28 years old, and the loss of hairs began when he was 51 years old.

His parents and his brothers had also white head hairs.

The patient rubbed onto his scalp the liniment according to example 1 in an amount of 15 ml once daily. After one month a part of his hair became black again and the loss of his hair decreased.

The treatment was continued for 2 further months. After this treatment period more than half of the patient's hair turned black. Additionally much new black hairs began to grow.

Conclusion

Example 2 shows the high therapeutic potential of the compositions of the present invention and their ability to
to prevent or at least to reduce the loss of hairs,
to restore the growth of the hairs and
to prevent or at least to reduce the formation of white hairs.

1. A composition for the treatment of the hair roots in order
to prevent or at least to reduce the loss of hairs,
to restore the growth of the hairs and
to prevent or at least to reduce the formation of white hairs,
characterized in that it contains an extract of at least one Chamomilla plant and/or of at least one Achillea plant.

2. The composition according to claim 1, characterized in that the extract comprises the oils, especially the essential oils, of the Chamomilla plant and/or of the Achillea plant.

3. The composition according to one of claims 1 to 2, characterized in that the extract is obtainable by steam distillation.

4. The composition according to one of claims 1 to 3, characterized in that the extract is obtainable from the flower heads of the Chamomilla plant and/or of the herbs and/or of the flower heads of the Achillea plant.

5. The composition according to one of claims 1 to 4, characterized in that the Chamomilla plant is selected from the group consisting of
Matricaria species, especially
Matricaria recutita L.
Matricaria discoidea DC.
Anthemis species, especially
Anthemis nobilis L.
Anthemis arvensis L.
Anthemis cotula L.
Anthemis tinctoria L.
Ormenis multicaulis Br.-BI & Maire
Eriocephalus punctulatus DC.,
whereby Anthemis nobilis L. is especially preferred.

6. The composition according to one of claims 1 to 5, characterized in that it contains at least one additional component selected from the group consisting of caffeine, theophylline, theobromine and folic acid.

7. The composition according to one of claims 1 to 6, characterized in that it contains at least one pharmaceutical and/or dermatological acceptable auxiliary agent.

8. The composition according to one of claims 1 to 7, characterized in that it is in such a form which is suitable
for topical application, especially shampoos, hair tonics, conditioners, pomades, lotions, sprays, liniments, creams, ointments, jellies, solutions suspensions or patches or
for oral application, especially in the form of capsules.

9. The composition according to one of claims 1 to 8, characterized in that it has a melanogenesis stimulating activity.

10. The composition according to one of claims 1 to 9, characterized in that the white hairs are the result of a condition selected from the group consisting of canities, ageing, poliosis and leukotrichia.

11. The composition according to one of claims 1 to 10, characterized in that the treatment comprises the step of applying or administering the composition to the surface of the skin, preferably on the scalp.

12. Use of a composition containing an extract of at least one Chamomilla plant and/or of at least one Achillea plant for the treatment of the hair roots in order
to prevent or at least to reduce the loss of hairs,
to restore the growth of the hairs and
to prevent or at least to reduce the formation of white hairs.

13. Use according to claim 12, characterized in that the composition is such a composition as defined in one of claims 2 to 11.

14. Use of a composition containing an extract of at least one Chamomilla plant and/or of at least one Achillea plant for the preparation of a medicament or of a cosmetic for the treatment of the hair roots in order
to prevent or at least to reduce the loss of hairs,
to restore the growth of the hairs and
to prevent or at least to reduce the formation of white hairs.

15. Use according to claim 14, characterized in that the composition is such a composition as defined in one of claims 2 to 11.

16. A method for the treatment of the human or animal body suffering under hair root problems, such as loss of hairs and formation of white hairs, characterized in that to the patient is applied or administered a composition containing an extract of at least one Chamomilla plant and/or of at least one Achillea plant.

17. The method according to claim 16, characterized in that the composition is such a composition as defined in one of claims 2 to 11.
